(19) United States
(12) Patent Application Publication
(10) Pub. No.: US 2021/0162108 A1
(43) Pub. Date: Jun. 3, 2021

(54) CARRYING DEVICE FOR BREAST MILK PUMP

(71) Applicant: Mary Spicciatie, Eden, NY (US)

(72) Inventor: Mary Spicciatie, Eden, NY (US)

(21) Appl. No.: 16/699,705

(22) Filed: Dec. 1, 2019

(51) Int. Cl. A61M 1/06; A61J 9/06; A45F 3/02

(52) U.S. Cl. CPC A61M 1/062; A61M 2210/1007; A45F 3/02; A61J 9/06

(57) ABSTRACT

An illustrated view of an exemplary carrying device for transportation and use to express breast milk is presented. The exemplary carrying device is useful for providing an easy method to transport a breast milk pump while keeping both hands free to attend to the child or other events. The carrying device is preferably made of a nylon cloth material. The shape of the carrying device is preferably an oblong shape. The carrying device is preferably a pouch. The carrying device has a strap for fitting over the shoulder and the strap is adjustable in length. Further, by using the strap the person's hands are free and can be used for other activities.

### FIELD OF THE INVENTION

This invention relates to carrying device. More particularly, it relates to a carrying device for a breast milk pump.

### BACKGROUND

Breast milk is the milk produced by the breasts (or mammary glands) of a human female to feed a child. Milk is the primary source of nutrition for newborns before they are able to eat and digest other foods; older infants and toddlers may continue to be breastfed, in combination with other foods from six months of age when solid foods should be introduced.

The baby nursing from its own mother is the most common way of obtaining breast milk, but the milk can be pumped and then fed by baby bottle, cup and/or spoon, supplementation drip system, or nasogastric tube. In preterm children who do not have the ability to suck during their early days of life, avoiding bottles and tubes, and use of cups to feed expressed milk and other supplements is reported to result in better breastfeeding extent and duration subsequently. Breast milk can be supplied by a woman other than the baby's mother, either via donated pumped milk (generally from a milk bank or via informal milk donation), or when a woman nurses a child other than her own at her breast, a practice known as wet nursing.

When a mother wishes to express breast milk, often times breast milk pumps are utilized. The breast milk pumps area often bulky and for privacy reasons a mother may not be able to just carry a breast milk pump and express milk in public or other locations. Therefore, there is a need for a device to transport breast milk pumps easily and where the mother has both hands available. There is also a need, to express mother's breast milk while not in a convenient location and provide a mechanism to provide privacy to the mother while utilizing the breast milk pump.

### BRIEF DESCRIPTION OF THE DRAWINGS

FIG. 1 is an illustrated view of an exemplary carrying device.

FIG. 2 is an illustrated view of the exemplary carrying device shown in FIG. 1 with the breast pump machine shown in FIG. 1.

### DETAILED DESCRIPTION

The phrases “in one embodiment,” “in various embodiments,” “in some embodiments,” and the like are used repeatedly. Such phrases do not necessarily refer to the same embodiment. The terms “comprising,” “having,” and “including” are synonymous, unless the context dictates otherwise. Such terms do not generally signify a closed list.

“Above,” “adhesive,” “affixing,” “any,” “around,” “both,” “bottom,” “by,” “comprising,” “consistent,” “customized,” “enclosing,” “friction,” “in,” “labeled,” “lower,” “magnetic,” “marked,” “new,” “nominal,” “not,” “of,” “other,” “outside,” “outwardly,” “particular,” “permanently,” “preventing,” “raised,” “respectively,” “reversibly,” “round,” “square,” “substantial,” “supporting,” “surrounded,” “surrounding,” “threaded,” “to,” “top,” “using,” “wherein,” “with,” or other such descriptors herein are used in their normal yes-or-no sense, not as terms of degree, unless context dictates otherwise.

Reference is now made in detail to the description of the embodiments as illustrated in the drawings. While embodiments are described in connection with the drawings and related descriptions, there is no intent to limit the scope to the embodiments disclosed herein. On the contrary, the intent is to cover all alternatives, modifications and equivalents. In alternate embodiments, additional devices, or combinations of illustrated devices, may be added to, or combined, without limiting the scope to the embodiments disclosed herein.

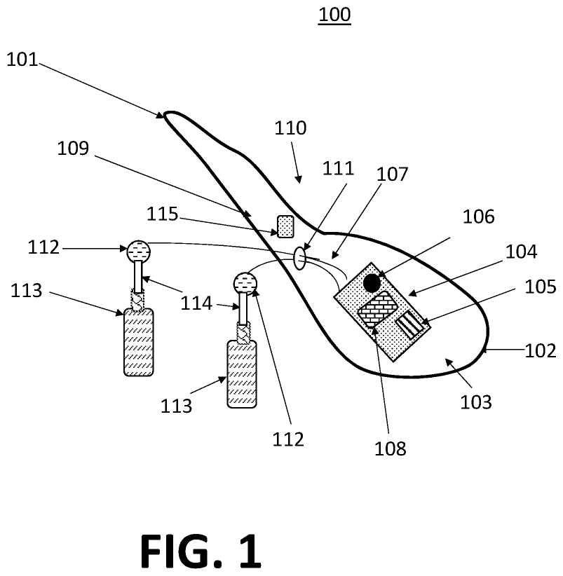

Referring to FIG. 1, an illustrated view of an exemplary carrying device 100, also known as “Pump on the Go,” for transportation and use to express breast milk is presented. The exemplary carrying device 100 is useful for providing an easy method to transport a breast milk pump 103 while keeping both hands free to attend to the child or other events. The carrying device 100 is preferably made of a stretchy nylon cloth material but other materials are hereby contemplated including, but not limited to, vinyl, cotton, plastic, etc. The shape of the carrying device 100 is preferably an oblong shape, but other shapes are hereby contemplated including, but not limited to, round, square, trapezoidal, octagonal, pentagonal, etc. The carrying device 100 is preferably a pouch, but other types of carrying devices are hereby contemplated, such as, but not limited to, a sling, a bag, etc. The carrying device 100 is configured to fit snuggly or tight to a person's body such that the carrying device 100 does not swing forward when the person bends over and remains snug to the body.

The carrying device 100 has a strap 101, a holding portion 102, a front 109, a pocket 115 and a back 110. The strap 101 of the carrying device 100 is preferably adjustable in length. The strap 101 of the carrying device 100 may have an adjustable coupling such as a buckle, a Velcro coupling, a button, a snap, etc. The strap 101 of the carrying device 100 is configured to fit over either a shoulder of a mother.

The holding portion 102 of the carrying device 100 has an interior portion 103. The interior portion 103 is a void area of the holding portion 102 of the carrying device 100. The interior portion 103 of the holding portion 102 of the carrying device 100 has a breast milk pump 104 and a port 111 through the back 110 of the carrying device 100. The breast milk pump 104 is useful for pumping breast milk from a mother.

The breast milk pump 104 has a power source 105, an actuator 106, one or more cables 107 and a motor 108. The power source 104 is preferably a rechargeable battery, but other types of power sources are hereby contemplated, including, but not limited to, replaceable batteries, solar, ac/dc power source, etc. The power source 105 being electrically coupled to the motor 108 of the breast milk pump 104.

The actuator 106 is preferably a button, but other types of actuators are hereby contemplated, including, but not limited to, toggle, wireless connectivity to a application on a computing device, etc. The actuator 106 for turning the breast milk pump 104 configured to control the electrical current from the power source 105 to the motor 108.

The motor 108 is communicatively coupled to one or more breast cups 112. The one or more breast cups 112 are removably coupled to a breast of a woman for expressing milk.

Each of the breast cups 112 are coupled to one of one or more bottles 113 by a connecting tube 114. The one or more bottles 113 for storing breast milk expressed by the breast cups 112 for storage and later use.

The pocket 115 is coupled to the front 109 of the carrying device 100. The pocket 115 is useful for carrying an item, such as a cell phone, tissues, wet wipes, etc. The cell phone 115 being carried in the pocket 115 has an application which is configured to communicate with the breast milk pump 104 and track usage and volume as well as a plethora of other details.

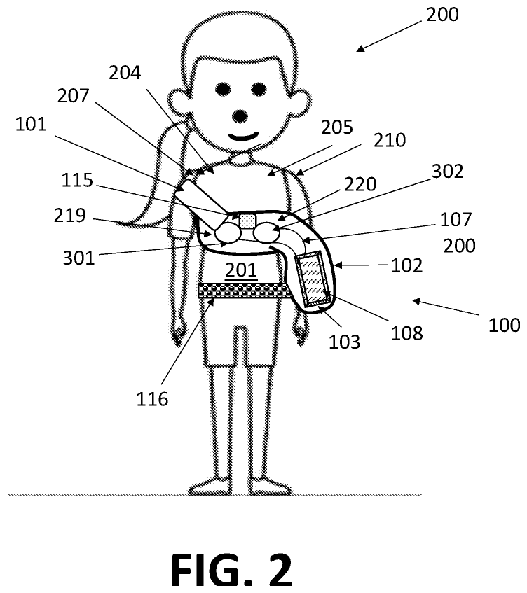

Moving now to FIG. 2, an illustrated view of the exemplary carrying device 100 shown in FIG. 1 is shown with being used to express breast milk into the one or more bottles 113 of FIG. 1. A mother 200 has a torso 201.

The torso 201 of the mother 200 has an upper right 204, an upper left 205 and a lower edge 206. The upper right 204 of the torso 201 of the mother 200 is coupled to a right shoulder 207.

The upper left 205 of the torso 201 of the mother 200 is coupled to a left shoulder 210. At least mid-way between the right shoulder 207 and the left shoulder 210 is a neck 216.

The strap 101 of the carrying device 100 is configured to fit over either the right shoulder 207 of the mother 200 or over the left shoulder 210 of the mother 200. Further, the holding portion 102 of the carrying device 100 is configured to be near either a right arm 208 of the mother 200 or a left arm 211 of the mother 200 depending on whether the mother 200 configures the position of the strap 101 of the carrying device 100 over the right shoulder 207 of the mother 200 or the left shoulder 210 of the mother 200.

The carrying device 100 further has a stabilizing strap 116. The stabilizing strap 116 is securely coupled to the holding portion 102 of the carrying device 100. The stabilizing strap 116 is preferably made of an elastic material, however other materials are hereby contemplated, including, but not limited to, a belt, a Velcro® strip, etc. The stabilizing strap 116 is further coupled to either a wrist or a waist of a wearer to prevent the carrying device 100 from swinging when bending over, walking, etc.

The breast milk pump 104 has a first breast cup 301 and a second pump 302. The first breast cup 301 and the second breast cup 302 are referenced in FIG. 1 as one or more breast cups 112. The first pump 301 is configured to be mechanically coupled to a first breast 219 of the torso 201 of the mother 200. The second pump 302 is configured to be mechanically coupled to a second breast 220 of the torso 201 of the mother 200.

When the breast pump 104 is actuated by the actuator 106 (shown in FIG. 1), the first breast cup 301 and/or the second breast cup 302 express breast milk from the first breast 219 and/or the second breast 220 of the mother 200. The connecting tube 114 (shown in FIG. 1) provides a coupling path between the breast cup 301 and one of the bottles 113 of the carrying device 100 to receive the expressed breast milk from the first breast 219 and/or the second breast 220.

The mother 200 may optionally have a cell phone configured in the pocket 115. The mother 200 can track the amount of breast milk and usage via an application configurable on the cell phone.

In the numbered clauses below, specific combinations of aspects and embodiments are articulated in a shorthand form such that (1) according to respective embodiments, for each instance in which a “component” or other such identifiers appear to be introduced (with “a” or “an,” e.g.) more than once in a given chain of clauses, such designations may either identify the same entity or distinct entities; and (2) what might be called “dependent” clauses below may or may not incorporate, in respective embodiments, the features of “independent” clauses to which they refer or other features described above.

Those skilled in the art will appreciate that the foregoing specific exemplary processes and/or devices and/or technologies are representative of more general processes and/or devices and/or technologies taught elsewhere herein, such as in the claims filed herewith and/or elsewhere in the present application.

The features described with respect to one embodiment may be applied to other embodiments or combined with or interchanged with the features of other embodiments, as appropriate, without departing from the scope of the present invention.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A carrying device for expressing breast milk, the device comprising:
a holding portion, the holding portion having a breast milk pump, wherein the breast milk pump for expressing breast milk;
a pocket, the pocket coupled to a front of the carrying device, wherein the pocket for carrying an item; and
a strap, the strap being configured to be coupled to a shoulder of a mother, wherein the mother's hands are free.

2. The device of claim 1, wherein breast milk pump further comprising:
a motor;
a power source, the power source for providing electrical energy to the motor;
an actuator, the actuator being coupled to the motor, wherein the actuator configured to control the electrical current to the motor’
one or more cables, the one or more cables coupled to one or more breast cups, the one or more breast cups being coupled to one or more breasts of the mother.

3. The device of claim 2, wherein the power source being a rechargeable battery.

4. The device of claim 2, wherein the actuator being a button.

5. The device of claim 2, wherein the actuator being an application on a computing device.

6. The device of claim 1, wherein the strap being configured to be coupled to a right shoulder of a mother.

7. The device of claim 1, wherein the strap being configured to be coupled to a left shoulder of a mother.

8. The device of claim 1, wherein the strap being adjustable in length.

9. The device of claim 1, wherein the strap having a buckle for adjusting the length of the strap.

10. The device of claim 1, wherein the strap having a snap for adjusting the length of the strap.

11. The device of claim 1, wherein the strap having a Velcro coupling for adjusting the length of the strap.

12. The device of claim 1, wherein the breast cups further comprising:
one or more connecting tubes, the connecting tubes being for carrying pumped breast milk; and
one or more bottles, the one or more bottles being coupled to the one or more breast cups by the one or more connecting tubes, and wherein the one or more bottles being for storage of the expressed breast milk.

13. The device of claim 1, wherein the device having an oblong shape.

14. The device of claim 1, wherein the device being made of a nylon cloth material.

15. The device of claim 1, wherein the device being a pouch.

16. The device of claim 1, wherein the device further has a stabilizing strap, wherein the stabilizing strap being coupled to the holding portion, and wherein the stabilizing strap being coupled to a waist or wrist of a person.

17. The device of claim 16, wherein the stabilizing strap being made of an elastic material.

18. The device of claim 1, wherein the device further comprising:
a back; and
a port, the port being configured on the back of the device, wherein the one or more cables being configured to be coupled to the one or more breast cups by exiting the device by the port.
